(19) United States
(12) Patent Application Publication
(10) Pub. No.: US 2007/0250176 A1
(43) Pub. Date: Oct. 25, 2007

(54) Hip Prosthesis with Anatomically Bent Shank

(75) Inventor: Sheila Ragbir, Whim (TT)

Correspondence Address: LAW OFFICES OF KARL HORMANN, 86 SPARKS STREET, CAMBRIDGE, MA, 02138, US

(73) Assignee: MARISAL INVESTMENT N.V., CURACAO (NL)

(21) Appl. No.: 11/659,349

(22) PCT Filed: Jun. 8, 2005
(86) PCT No.: PCT/IB05/01835

§ 371 Date: Feb. 5, 2007

(30) Foreign Application Priority Data
Aug. 6, 2004 (CH) 01310/04

(51) Int. Cl. A61F 2/42

(52) U.S. Cl. 623/23.15

(57) ABSTRACT

A hip prosthesis (1) comprising a shank (2) which is inserted into the extremity of the femur (3), in which the longitudinal axis (H-H) of the shank (2) follows a path substantially parallel to the external profile (P) of the head of the femur (3) in the top part of its cervical-trochanter area (3a) is described.

This invention relates to the technological sector dedicated to the manufacture of hip prostheses.

As is known, these prostheses comprise a shank bearing the joint prosthesis proper which is inserted into the patient's femur.

This operation, using existing hip prostheses, is carried out by coaxially inserting the shank into the diaphysis of the femur after the entire head and neck of the same have been amputated.

The inventor of this invention is of the opinion that this type of surgery is in many cases more destructive than is necessary, sacrificing the diaphysis bone and in particular the metaphysis part thereof, restricting or thus rendering more complex the possibility of subsequent limb prosthesis surgery.

In order to overcome these disadvantages the inventor has conceived of a hip prosthesis with the shank formed in such a way that it can be inserted transversely into the neck of the femur after resection of the epiphysis alone has been performed.

The shank of the hip prosthesis according to the invention is in fact designed in such a way that it can be inserted into the proximal part of the femur, causing it to pass through the cervical and large trochanter area, that is particularly within the cervical-trochanter area, without having to insert it coaxially into the diaphysis.

In order to achieve this objective the inventor has conceived a hip prosthesis which comprises a shank whose longitudinal axis follows a curved path substantially similar to the profile of the top part of the femur in the said cervical-trochanter area.

As will be better seen below, the aforesaid longitudinal axis may comprise a curved section substantially reproducing the form of the said external profile of the cervical-trochanter area, or at least two consecutive straight segments not in line which approximately match the said shape substantially, with an upwards facing concavity.

In both cases the longitudinal axis of the aforesaid shaft is, when inserted in the femur, approximately parallel to the profile of the cervical-trochanter area already described.

The subject matter of this invention therefore comprises a hip prosthesis as described in appended Claim 1.

A more detailed description of two preferred embodiments of the hip prosthesis according to the invention will now be provided with reference to the appended drawings, in which:

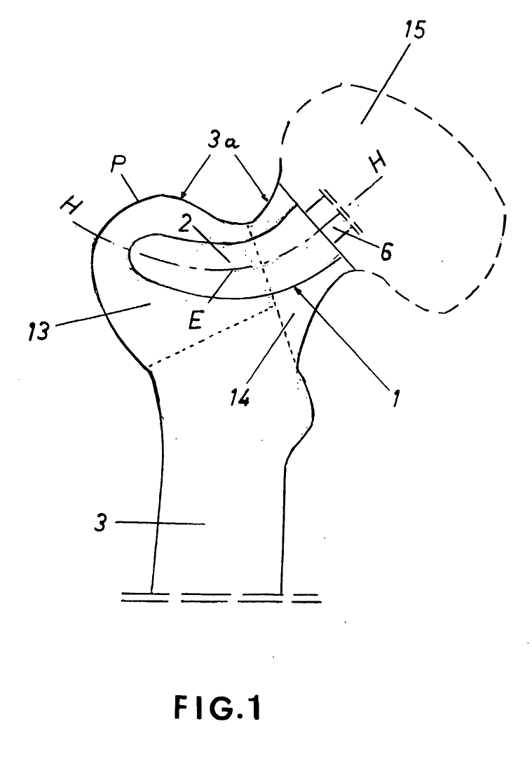

FIG. 1 is a diagrammatical longitudinal cross-section of part of a femur in which a prosthesis according to the invention has been inserted in which the longitudinal axis of the shank follows a curving path with an upward concavity,

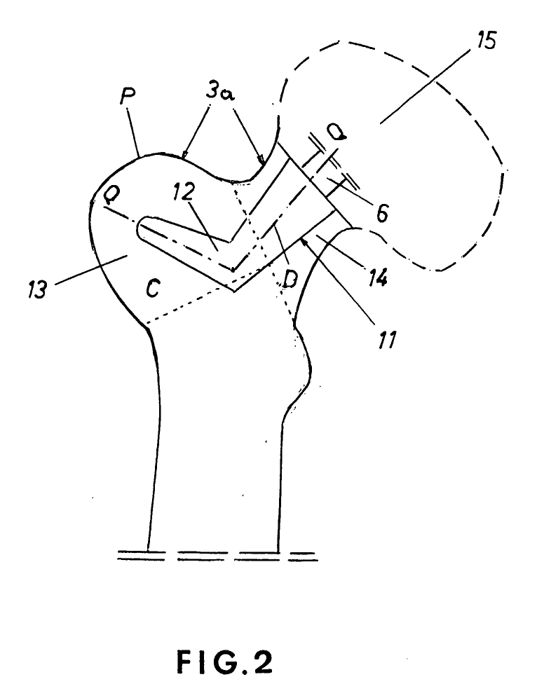

FIG. 2 is a diagrammatical longitudinal cross-section of part of a femur in which a prosthesis according to the invention is inserted in which the longitudinal axis of the shank comprises two consecutive segments not in line which form an upward concavity,

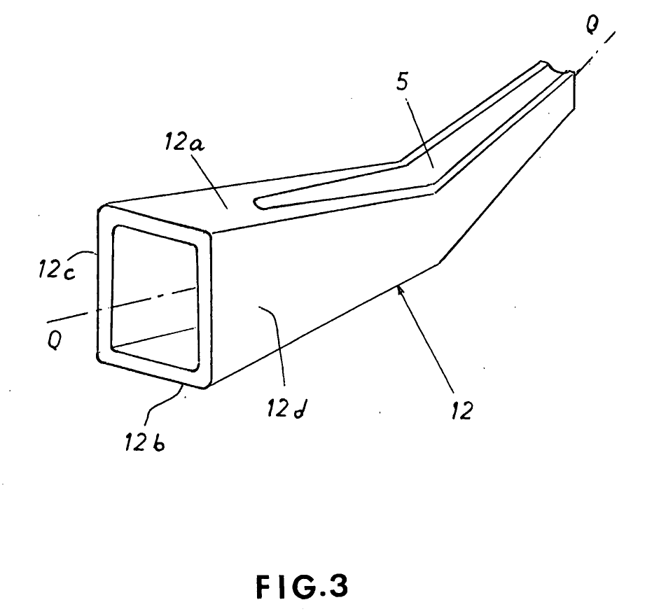

FIG. 3 is a perspective view of the hip prosthesis of the type of that in FIG. 2, with a recess for better anchoring in the bony tissue.

Considering first FIG. 1, this shows how a hip prosthesis 1 according to the invention comprises a shank 2 to which there is connected a collar 6 of the modular type (only referred to in the drawing) designed to support a spherical head (not shown). Said shank 2 is shaped in such a way that its longitudinal axis H-H comprises a curved length E, the shape and upward concavity of which is substantially similar to the outer profile P of the head of the femur 3 in the top part of its cervical-trochanter area 3a, which as is known comprises the cervical area 13 and the area of the great trochanter 14. Shank 2 thus runs transversely through the neck 13 of the femur remaining substantially parallel to the said profile P, and in order for it to be fitted it is only necessary to move epiphysis 15, indicated in the figure by a dashed line.

Using a hip prosthesis 1 according to the invention it is therefore possible to achieve a reliable implant in the femur 3 of the patient without it being necessary to act destructively removing the metadiaphysis tissue, and this offers the advantages already illustrated above.

FIGS. 2, 3 illustrate another embodiment of a hip prosthesis 11 according to the invention, for which the same considerations as stated in the description of the previous case apply.

The only difference lies in the fact that in prosthesis 11 in question shank 12 is shaped in such a way that its longitudinal axis Q-Q comprises two consecutive straight segments C, D which are not in line with each other.

Again in this case this longitudinal axis can be arranged in such a way that it runs approximately parallel to the abovementioned profile P, with the same results and the same advantages.

Of course, if it is desired to make the path of the aforesaid longitudinal axis even more similar to the profile P of the head of the femur a prosthesis provided with a shaft according to the invention in which the said longitudinal axis comprises a piece formed of a number of consecutive unaligned segments greater than 2 may be constructed. (This situation is not shown in the drawings).

In FIG. 3 it will be seen how, advantageously, the inventor suggests that shank 12 of the prosthesis according to the invention may be constructed having a square quadrangular cross-section comprising four faces in two parallel pairs 12a, 12b, 12c, 12d, in order to achieve the known advantages of resistance to torsional stresses.

The inventor also provides the possibility of forming a recess 5 in the concave part of shank 12, which in this case comprises surface 12a in the top position, extending approximately the entire length of the shank in order to improve the reliability of the connection between the prosthesis and the bony tissue of the femur in a known way.

1. Hip prosthesis (1, 11), comprising a shank (2, 12) which is inserted into the extremity of the femur (3), characterized in that the longitudinal axis (H-H, Q-Q) of the said shank (2, 12) has a path substantially parallel to the external profile (P) of the head of the femur (3) in the top part of its cervical-trochanter area (3a).

2. Hip prosthesis according to claim 1, characterized in that the said longitudinal axis (H-H) of the shank (2) comprises a curved section (E) having concavity at the top.

3. Hip prosthesis according to claim 1, characterized in that the said longitudinal axis (Q-Q) of the shank (12) comprises at least two consecutive straight segments (C, D) which are not in line.

4. Hip prosthesis according to claim 1 characterized in that the said shank (12) has a square quadrangular cross-section comprising four faces in two parallel pairs (12a, 12b, 12c, 12d).

5. Hip prosthesis according to claim 1, characterized in that a recess (5) which extends around almost the entire length of the said shank (2) is formed in the concave surface of the said shank (2).

6. Hip prosthesis according to claim 2, characterized in that the said shank (12) has a square quadrangular cross-section comprising four faces in two parallel pairs (12a, 12b, 12c, 12d).

7. Hip prosthesis according to claim 3, characterized in that the said shank (12) has a square quadrangular cross-section comprising four faces in two parallel pairs (12a, 12b, 12c, 12d).

8. Hip prosthesis according to claim 2, characterized in that a recess (5) which extends around almost the entire length of the said shank (2) is formed in the concave surface of the said shank (2).

9. Hip prosthesis according to claim 3, characterized in that a recess (5) which extends around almost the entire length of the said shank (2) is formed in the concave surface of the said shank (2).

10. Hip prosthesis according to claim 4, characterized in that a recess (5) which extends around almost the entire length of the said shank (2) is formed in the concave surface of the said shank (2).
